(19) United States
(12) Patent Application Publication
(10) Pub. No.: US 2025/0134561 A1
(43) Pub. Date: May 1, 2025

(54) TWO-PART INTRAMEDULLARY NAIL, AND NAIL CORE THEREFOR

(71) Applicant: JABEZ MEDICAL GMBH & CO., KG, Glattbach (DE)

(72) Inventor: Siegbert Gregel, Edertal (DE)

(21) Appl. No.: 18/710,261

(22) PCT Filed: Nov. 15, 2022
(86) PCT No.: PCT/DE2022/100854

§ 371 Date: Sep. 5, 2024

(30) Foreign Application Priority Data
Nov. 16, 2021 (DE) 20 2021 106 260.6

(51) Int. Cl. A61B 17/72

(52) U.S. Cl. CPC A61B 17/7233

(57) ABSTRACT

In an embodiment an intramedullary nail includes a sheath element extending in a longitudinal direction and having a proximal end, a distal end, and a substantially cylindrical interior, wherein an external contour of the sheath element, viewed perpendicularly to the longitudinal direction, includes an elongate spring element, which is wound in a helical shape starting from the proximal end and with respect to the longitudinal direction and wherein an extension of the external contour of the sheath element is reducible by tensioning the elongate spring element in a winding direction and is increasable by detensioning the elongate spring element counter to the winding direction, and a substantially rod-shaped nail core insertable into the interior through the proximal end of the sheath element, wherein the sheath element and the rod-shaped nail core are two separate parts.

### CROSS-REFERENCE TO RELATED APPLICATIONS

This patent application is a national phase filing under section 371 of PCT/DE2022/100854, filed Nov. 15, 2022, which claims the priority of German utility model application 20 2021 106 260.6, filed Nov. 16, 2021, each of which is incorporated herein by reference in its entirety.

### TECHNICAL FIELD

The invention relates to a two-part intramedullary nail for the internal stabilization and/or fixing of an elongate hollow body, in particular a bone, and to a nail core therefor.

### BACKGROUND

Techniques and devices for intramedullary nailing in case of bone fractures of the extremities were first significantly developed and described by Küntscher et al., see Küntscher, G., Maatz, R., “Technik der Marknagelung”, Georg Thieme Verlag Leipzig, 1945.

Simpler intramedullary nails for interlocking nailing are known, but their installation during surgery can only be checked by means of intraoperative X-ray imaging. This is, due to the duration, stressful not only for the patient but in particular for the surgical staff. Furthermore, such simple intramedullary nails must be secured in the bone using transverse screws, which requires complex targeting devices, in particular for long femurs, but also for humeri and tibiae, in order to be able to place such transverse screws precisely through the skin with as little damage to the skin as possible. These additional surgical wounds that have to be inflicted into an extremity have to be treated after surgery, but also after surgical removal of the transverse screws, which causes additional stress for the patient.

DE 197 31 298 A1 discloses a nail system for intramedullary osteosynthesis in all types of shaft fractures, which comprises a tubular outer body with openings from which hooks extend outwards, which can be pivoted outwards by actuating an inner body, whereby the hooks can contact the inner wall of a tubular bone. This nail system is complex and comprises many components, but owing to its principle it can only establish point contacts with the inner surface of a tubular bone, and establish said contacts only in a narrow circumferential area that is identical over its length.

DE 38 35 682 A1 discloses a device for stabilizing tubular bone fractures and joints, which comprises helical springs wound into one another in a simplex or multiplex way as stabilizing elements, some of which are wound around a core in the form of an elastically deformable wire or a bundle of elastically deformable wires. The ends of such a helical spring can be configured with a device for fixing to the bone.

WO 2006/066536 A1 discloses an intramedullary nail, in particular for the closed reposition of a fracture of a tubular bone by insertion into a drill hole in its medullary cavity, which is of comparatively simple construction and can be used easily, quickly and safely with only minimal use of intraoperative X-rays, without requiring interventions in unhurt areas of an extremity. The intramedullary nail comprises a nail core, at the front region of which a head piece is provided, at which the origin of a strip is fixed. The strip is wound around the nail core and is fixed at its end in the rear region around the nail core in a rotationally lockable manner, and the strip expands the radial contour of the intramedullary nail when it is rotated in the opposite direction to the direction in which it is wound.

Advantageous further developments of this intramedullary nail with a nail core are the subject of publications WO 2014/060576 A1 and WO 2014/060578 A1.

When implanting intramedullary nails with solid or rigid nail cores or cores, the problem is to insert the intramedullary nail correctly from a lateral insertion point into the essentially straight medullary canal. For example, in the shank, there exists a very narrow kink into the medullary canal, which in turn extends straight. A corresponding kink in the intramedullary nail is known as a Herzog bend. This kink can be approx. 10° to the straight axis, for example.

With the aforementioned intramedullary nails with variable diameters, the problem arises that the fixed nail core limits a reduction in the diameter of the entire intramedullary nail, so that implantation is also made more difficult here, in particular in case of a kink between the insertion point and the medullary canal.

WO 2015/117996 A1 discloses a further intramedullary nail, namely an assembly for the internal stabilization and/or fixing of an elongate hollow body, in particular a bone, such as a tubular bone, or a prosthesis, with a stabilizing element with an elongate first element comprising a helical shape, the proximal end of which is connected to a first fastening element, and from the distal end of which a second element extends, which is connected to a second fastening element in the region of the first fastening element, wherein the first element and the second element preferably merge into one another connectionless as sections of the stabilizing element, wherein an outer fastening element forming the first fastening element clampingly fixes an inner fastening element forming the second fastening element, so that the first fastening element cannot be twisted relative to the second fastening element. In this assembly with two wound elements lying one above the other, no inner nail core is provided.

Other comparable items are shown in DE 10 2018 005 690 A1 and DE 20 2020 003 051 U1.

Such intramedullary nails without an inner nail core or core are very pliable and flexible and can therefore be implanted easily. Insertion through a narrow kink into an intramedullary canal also hardly causes problems. These intramedullary nails can be considerably reduced in diameter by tensioning the fastening elements without a nail core getting in the way. However, intramedullary nails of this type comprise only a low bending stiffness and only limited lateral and axial stability.

### SUMMARY

Embodiments provide an intramedullary nail which, on the one hand, can be easily implanted even in confined or complicated spaces and, on the other hand, exhibits a high stability against bending and lateral and axial stress after implantation.

Embodiments provide an intramedullary nail for internal stabilization and/or fixing of an elongate hollow body, in particular a bone, wherein the intramedullary nail comprises two separate parts, namely
a) a sheath element extending in a longitudinal direction and having a proximal end, a distal end, and a substantially cylindrical interior, wherein an external contour of the sheath element, viewed perpendicularly to the longitudinal direction, comprises an elongate spring element, which is wound in a helical shape starting from the proximal end and with respect to the longitudinal direction, and wherein the extension of the external contour of the sheath element can be reduced by tensioning the elongate spring element in the winding direction and can be increased by detensioning the elongate spring element counter to the winding direction, and
b) a substantially rod-shaped nail core which can be inserted into the interior through the proximal end of the sheath element.

Within the scope of the invention, it is possible to create a stability in intramedullary nails which, on the one hand, allows problem-free implantation even through a bend or a tight kink and, on the other hand, provides high stability after implantation.

The separate nail core can be subsequently inserted into the already implanted sheath element, where it can provide significantly increased bending stability as well as lateral and axial stability of the entire intramedullary nail. On the other hand, implantation through an incision point, in particular through a narrow kink, is not hindered by a stiff nail core.

In this case, the nail core is an independent part of the entire intramedullary nail and is not connected to the sheath element until implantation. This makes the implantation procedure particularly flexible and adaptable to a wide variety of spatial conditions.

The sheath element can be compressed particularly tightly without a contained nail core, namely by tensioning the spring element, and can therefore be inserted into a medullary canal with absolute flexibility and pliability.

The implanted sheath element can then act as a channel for the nail core to be inserted subsequently. The nail core can slide particularly well on the inner wall of the sheath element and can therefore be inserted more easily into the hollow body or bone.

Depending on the implantation site, the diameter and length of the nail core can be selected in a ratio that allows sufficient flexibility to guide the core through tight radii, and that also provides sufficient stability for the entire intramedullary nail.

In the following, the aspects according to the invention are discussed further, for which purpose reference is made in part to non-limiting advantageous embodiments and further developments of the invention. The features of advantageous further developments can be realized individually or in any combination, whereby further advantageous embodiments of the invention are created.

Throughout the present discussion of configurations, further developments, examples and embodiments of the devices according to the invention, methods of using the devices and uses of the devices according to the invention are also described. These methods and uses also expressly lie within the scope of the present invention.

In a first preferred further development of the intramedullary nail according to the invention, the sheath element essentially has the shape of a hollow cylinder. In this way, the sheath element can be easily inserted into a hollow body to be stabilized, and at the same time a defined interior is formed for the nail core.

Preferably, the external contour of the sheath element is essentially formed by the wound, elongated spring element.

Preferably, the spring element is wound in the form of a three-dimensional coil, and in particular in the form of a cylindrical coil.

In another advantageous embodiment, the spring element is elastically deformable.

The spring element can preferably form a three-dimensional coil spring, and in particular a helical spring.

There may be a gap or no gap between adjacent windings of the spring element. If adjacent windings lie directly on top of each other, a situation is usually reached in which adjacent windings exert a considerable static friction force against each other.

The spring element preferably is a wire or a strip.

Another advantageous design of the intramedullary nail is one in which the sheath element is, by expanding its external contour, braceable against the inner wall of an elongate hollow body to be stabilized. In this way, the sheath element can be firmly anchored in the bone after implantation and at the same time stabilize and, if necessary, align the fracture. Furthermore, the sheath element braced against the inner wall provides a maximally large interior for the nail core.

The intramedullary nail can preferably comprise a sheath element with two elongate spring elements arranged one above the other, and which are each wound in a helical shape with respect to the longitudinal direction of the sheath element.

The two spring elements arranged one above the other can preferably form a three-dimensional double spiral, in particular a double helix.

The two spring elements mentioned above, arranged one above the other, can be wound in opposite directions or in the same direction.

A particularly preferred configuration of the intramedullary nail is one in which the two spring elements arranged one above the other are wound in opposite directions, and consist of a one continuous element which, at the distal end, transitions from a first spring element into a second spring element.

Preferably, the nail core is insertable from the outside into a sheath element which itself is already inserted into a hollow body to be stabilized, or which itself is already implanted in a bone.

With regard to facilitating an insertion of the intramedullary nail into the sheath element, it is preferable that the nail core is elastically deformable, in particular elastically bendable.

According to a further advantageous embodiment, the nail core is insertable into a sheath element which comprises a kink or a bend. In this respect, it is particularly advantageous if the intramedullary nail is elastically deformable.

The nail core can preferably be made of solid material.

Preferably, the nail core comprises a round, in particular circular, cross-section. This means that the nail core can be easily inserted and advanced into the sheath element, and at the same time the nail core can comprise a similar or the same bending stiffness in relation to all directions perpendicular to the longitudinal direction.

To further simplify implantation, the nail core can preferably comprise a Herzog curvature.

A particularly preferred embodiment is one in which the proximal end of the sheath element comprises a substantially axial aperture for insertion of the nail core.

Preferably, the proximal end of the sheath element comprises a nail head. Such a nail head can, in particular, provide an anchorage for the origin or the end of a spring element.

The nail head can preferably comprise a sleeve or bushing which is rotatable about the longitudinal axis and which is connected to the origin or end of a spring element. The respective spring element can then be tensioned or detensioned by rotating the sleeve or bushing about the longitudinal axis.

Further preferably, the proximal end of the sheath element, in particular a nail head, may comprise an end cap on the side facing away from the sheath element. In particular, this allows a nail core inserted into the sheath element to be covered and further secured. Furthermore, this results in a hygienic, smooth surface of the proximal end, in particular of the nail head, which is well tolerated by the adjacent tissue and bone material.

Preferably, the length of the nail core correlates with the axial extension of the wound spring element plus the length of the nail head.

Preferably, the distal end of the nail core comprises a tip that is rounded. This makes it particularly easy to insert the nail core into and through the sheath element.

It proves to be further advantageous if the proximal end of the nail core is fixable or anchorable in the proximal end of the sheath element, in particular in a nail head.

The proximal end of the nail core is particularly preferably widened compared to another radial extension of the nail core. In particular, the widened end can serve as a stop to anchor the proximal end in the sheath element or in a nail head. This prevents the nail core from migrating through the sheath element. In the worst-case scenario, the nail core could otherwise permanently work its way through the bone and damage an adjacent joint.

A widening of the nail core can preferably be realized by the process of forming, impacting or crushing, or by a thread.

In a further advantageous embodiment, a diameter of the sheath element is reducible to a diameter of the nail core by tensioning a spring element.

An advantageous embodiment of the intramedullary nail according to the invention also relates to a design in which the nail core is already inserted into the interior of the sheath element.

Further embodiments provide a method of internally stabilizing and/or fixing an elongate hollow body, the method comprising the following steps, and in particular comprising the steps in the order listed:
a) Providing an intramedullary nail according to the invention,
b) Optionally, reducing the extension of the external contour of the sheath element by tensioning the elongate spring element in the winding direction,
c) Inserting the sheath element into the hollow body, starting with its distal end,
d) Optionally, increasing the extension of the external contour of the sheath element by detensioning the elongate spring element counter to the winding direction,
e) Optionally, bracing the sheath element against an inner wall of the hollow body by further detensioning the elongate spring element counter to the winding direction,
f) Inserting, starting with its distal end, the nail core into the interior of the sheath element via the proximal end of the sheath element,
g) Optionally, anchoring the nail core in the sheath element, in particular in a nail head.

Finally, embodiments provide a method of internally stabilizing and/or fixing a bone, the method comprising the following steps, and in particular comprising the steps in the order listed:
a) Providing an intramedullary nail according to the invention,
a1) Optionally, creating an incision point in the bone,
a2) Optionally, drilling out or milling out the medullary canal,
b) Optionally, reducing the extension of the external contour of the sheath element by tensioning the elongate spring element in the winding direction,
c) Inserting, starting with its distal end, the sheath element into the bone, in particular via an incision point in the bone,
d) Optionally, increasing the extension of the external contour of the sheath element by detensioning the elongate spring element counter to the winding direction,
e) Optionally, bracing the sheath element against an inner wall of the bone by further detensioning the elongate spring element counter to the winding direction,
f) Inserting, starting with its distal end, the nail core into the interior of the sheath element via the proximal end of the sheath element,
g) Optionally, anchoring the nail core in the sheath element, in particular in a nail head,
g1) Optionally, closing the proximal end of the sheath element.

The proposed methods also serve to further understand and illustrate the teaching according to the invention.

### BRIEF DESCRIPTION OF THE DRAWINGS

Supplementary or in addition to the advantageous embodiments and further developments of the teaching already discussed, embodiments of devices, uses and methods according to the invention shown in the drawings to the extent of FIGS. 1 to 6 are explained in more detail below. However, the examples discussed with reference to the drawings do not limit the invention to the examples shown. In discussing the embodiments with reference to the drawing, generally preferred embodiments and further developments of the teaching are also shown.

Further developments of the advantageous embodiments described above with features of the embodiments described below, just like further developments of the embodiments described below with features of the embodiments described above, expressly form further advantageous embodiments of the invention, and thus form part of the present disclosure.

With regard to the following explanation of the figures, it should generally be noted that, for reasons of clarity, reference signs already shown in previous figures and already explained above in this respect have in some cases not been repeated in subsequent figures and/or are in some cases not explained again on the basis of these subsequent figures. For the explanation of such reference signs and the associated technical features, reference is therefore made in full to the respective description of the corresponding preceding figures in order to avoid repetition.

FIG. 1 shows a lateral view of a preferred embodiment of the intramedullary nail according to the invention, shown here with the nail core not fully inserted;

FIG. 2 shows a lateral view of the embodiment from FIG. 1, shown here with a curved run;

FIG. 3 shows a lateral view of the nail core of the embodiment shown in FIGS. 1 and 2;

FIG. 4 shows a lateral and enlarged sectional view of the nail head of the embodiment shown in FIGS. 1 and 2, shown here with the nail core fully inserted;

FIG. 5 shows a perspective view of the embodiment from FIGS. 1 and 2, shown here with the nail core fully inserted; and

FIG. 6 shows a perspective view of the embodiment from FIGS. 1, 2 and 5, shown here with the nail core fully inserted, and with an end cap positioned above it.

### DETAILED DESCRIPTION OF ILLUSTRATIVE EMBODIMENTS

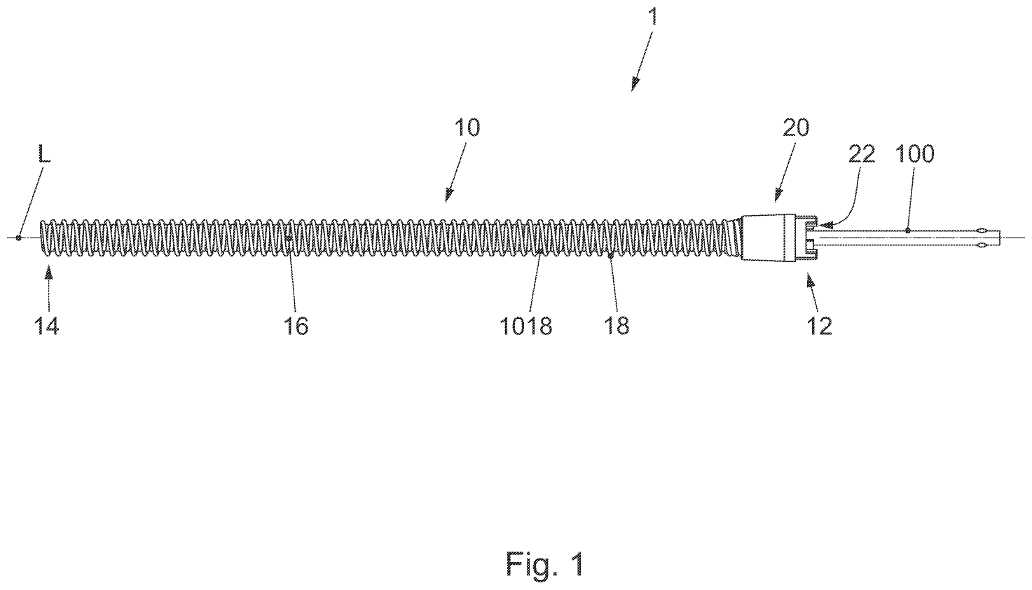

FIG. 1 shows a lateral view of a preferred embodiment of the intramedullary nail 1 according to the invention, comprising a sheath element 10 and a nail core 100, wherein here, for illustration purposes, the nail core 100 is not shown fully inserted into the sheath element 10.

The intramedullary nail 1 according to the invention comprises two separate parts, namely a sheath element 10 extending in a longitudinal direction L, with a proximal end 12, a distal end 14, and with a substantially cylindrical interior 16, wherein an external contour of the sheath element 10 as seen perpendicularly to the longitudinal direction L comprises an elongate spring element 18 which, starting from the proximal end 12 and with respect to the longitudinal direction L, is wound in a helical shape.

The extension of the external contour of the sheath element 10 can be decreased by tensioning the elongate spring element 18 in the winding direction, and can be increased by detensioning the elongate spring element 18 counter to the winding direction.

Furthermore, the intramedullary nail 1 comprises a substantially rod-shaped nail core 100, which can be inserted or introduced into the interior 16 through the proximal end 12 of the sheath element 10.

For this purpose, the sheath element 10 essentially has the shape of a hollow cylinder, so that the interior 16 essentially assumes a cylindrical shape.

Here, the spring element 18 essentially conceals the view of the interior 16, as the external contour of the sheath element 10 is essentially formed by the wound spring element 18.

The spring element 18 is wound in the form of a three-dimensional coil, and here in particular in the form of a cylindrical coil.

Furthermore, the spring element 18 is elastically deformable, therefore it can be assumed that the spring element 18 here forms a three-dimensional wound spring, and here in particular a helical spring. Furthermore, the spring element 18 here is an elastically deformable wire or spring wire.

There is a gap between adjacent windings of the spring element 18. This means that the diameter of the sheath element 10 can be easily reduced by tensioning the spring element 18. On the other hand, the sheath element 10 can also be braced relative to the inner wall of an elongate hollow body to be stabilized by extending its external contour, namely by detensioning the spring element 18.

In this embodiment, the sheath element 10 comprises not just one spring element 18, but two elongate spring elements 18, 1018 arranged one above the other, which are each wound in a helical shape in relation to the longitudinal direction L of the sheath element 10. To this end, a second spring element 1018 extends within the spring element 18 or first spring element 18.

Here, the two spring elements 18, 1018 arranged one above the other form a three-dimensional double spiral, namely in particular a double helix.

The two spring elements 18, 1018 arranged one above the other are here wound in opposite directions compared to each other, and consist of one continuous element which transitions from the first spring element 18 into the second spring element 1018 at the distal end 14.

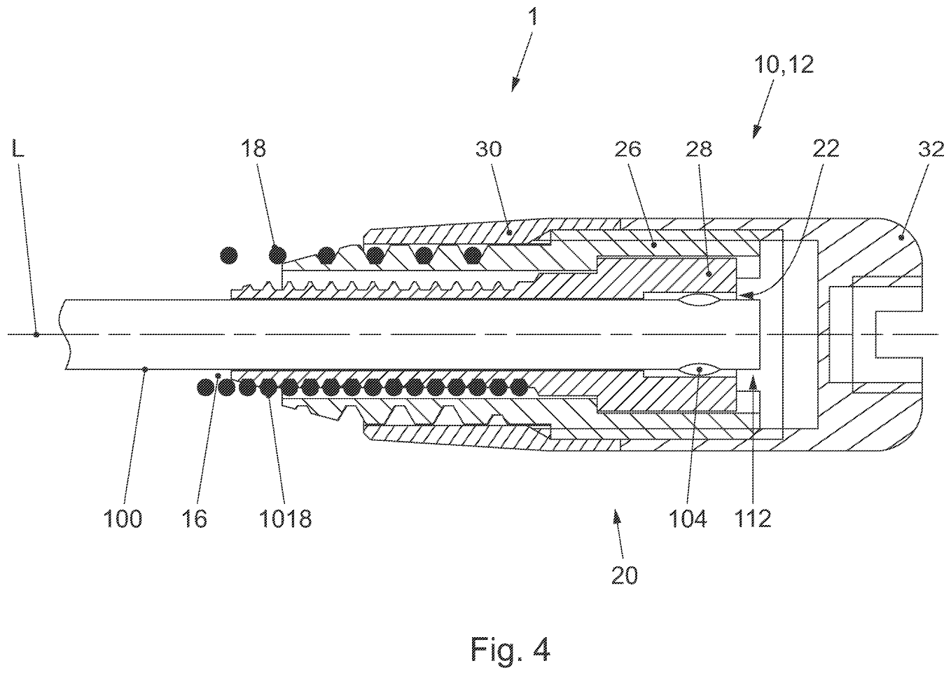

The proximal end 12 of the sheath element 10 comprises a nail head 20. The origin of the first spring element 18 and the end of the second spring element 1018 are arranged on the nail head 20. Furthermore, the nail head 20 comprises an axial aperture 22 for inserting the nail core 100. Further details of the nail head 20 will be discussed with reference to FIG. 4.

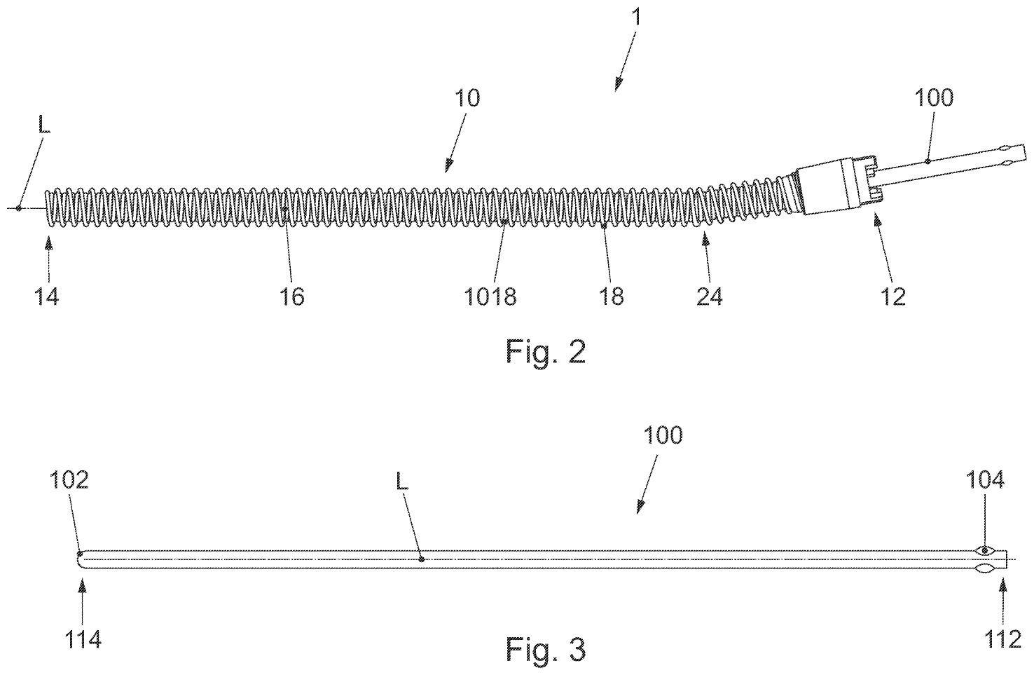

FIG. 2 shows a lateral view of the embodiment of the intramedullary nail 1 according to the invention from FIG. 1, whereby a curved run of the intramedullary nail 1 is shown here.

It becomes clear from this figure that the nail core 100 can also be inserted from the outside into a sheath element 10 which itself is already inserted into a hollow body to be stabilized (not shown), or which itself is already implanted in a bone (not shown).

In particular, the nail core 100 can also be inserted into a sheath element 10 comprising a kink 24 or a bend. For this purpose, the nail core 100 is designed to be elastically deformable, here in particular elastically bendable.

The kink 24 in the sheath element 10 here amounts to approx. 10° relative to the longitudinal direction L. Such a kink 24 arises in particular during implantation into an incision point which is arranged laterally with respect to the medullary canal, for example in a lower leg bone.

FIG. 3 shows a lateral view of the nail core 100 of the embodiment of the intramedullary nail 1 according to the invention shown in FIGS. 1 and 2.

The nail core 100 is made of solid material and has a circular cross-section.

The distal end 114 of the nail core 100 comprises a tip 102 which is rounded. This makes it much easier to insert and pass the nail core 100 into the already implanted sheath element 10.

Furthermore, the proximal end 112 of the nail core 100 is widened with respect to the remaining radial extension of the nail core 100. This allows the nail core 100 to be anchored with its proximal end 112 in the nail head 20.

This widening 104 of the proximal end 112 of the nail core 100 is realized by suitable forming, for example crushing, of the proximal end 112.

FIG. 4 shows a lateral and enlarged sectional view of the nail head 20 of the embodiment of the intramedullary nail 1 according to the invention shown in FIGS. 1 and 2, which is shown here with the nail core 100 fully inserted.

This illustration shows the centrally arranged nail core 100 which has been completely inserted into the interior 16 of the sheath element 10.

An outer sleeve 26 rotatable about the longitudinal direction L is fastened to the origin of the first spring element 18. By turning the outer sleeve 26 in the winding direction of the spring element 18, the spring element is further tensioned and the diameter of the sheath element 10 is reduced.

A further inner sleeve 28 rotatable about the longitudinal direction L is fastened to the end of the second spring element 1018. By turning the inner sleeve 28 counter to the winding direction of the second spring element 1018, the spring element 1018 is further detensioned, and can finally bear attach to the inside of the first spring element 18 in order to stabilize and secure it.

The outer locking sleeve 30 secures the outer and first spring element 18.

The proximal end 112 of the nail core 100 is anchored in the inner sleeve 28 or in the axial aperture 22 due to the widening 104 of its proximal end 112. Owing to the widening 104, the nail core 100 cannot move any further towards the distal end 14 (not shown here) of the sheath element 10. Otherwise, in an unfavorable case, the nail core 100 could gradually migrate through the sheath element 10 into an adjacent bone or joint and cause damage there.

Finally, an end cap 32 is assembled over the proximal end 112 of the nail core 100, which can be screwed onto the proximal end 12 of the sheath element 10, in this case onto the right side of the nail head 20. The end cap 32 prevents an undesired migration of the nail core 100 from the sheath element 10, namely via its proximal end 12. Furthermore, the end cap 32 provides an outwardly smooth surface of the intramedullary nail 1, which is thus well tolerated by the surrounding tissue or bone material.

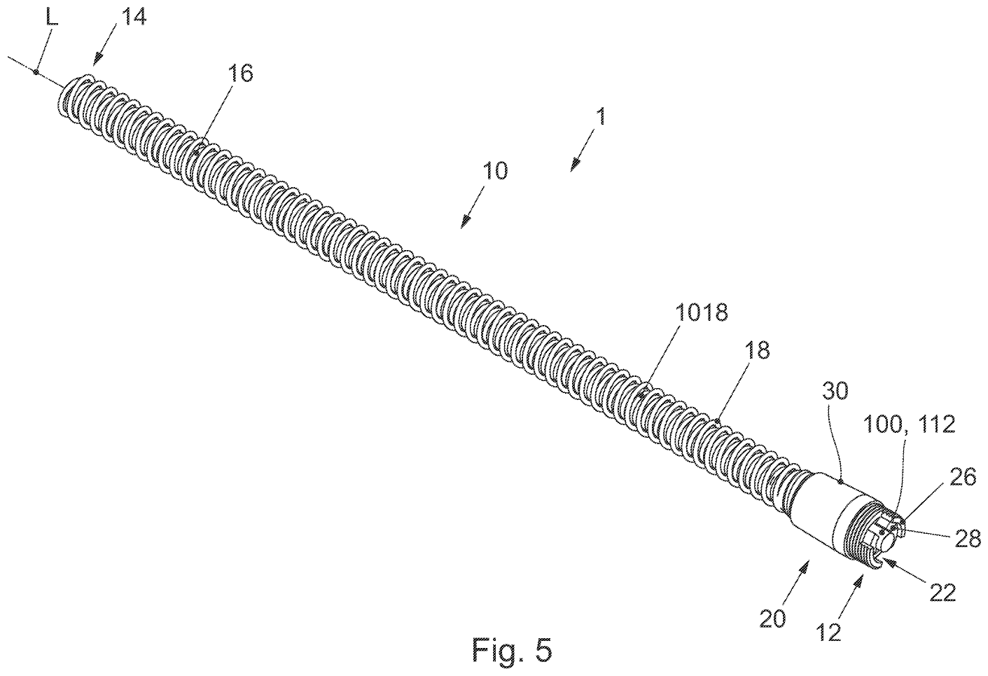

FIG. 5 shows a perspective view of the embodiment of the intramedullary nail 1 according to the invention from FIGS. 1 and 2, which is shown here with the nail core 100 fully inserted.

The spring elements 18, 1018 arranged one above the other in the form of a double helix are particularly easy to recognize here.

Furthermore, the axial aperture 22 in the proximal end 12 of the sheath element 10, namely here in the nail head 20, can be clearly seen in this illustration. Viewed from the inside to the outside, the inner sleeve 28, the outer sleeve 26 and the locking sleeve 30 enclose the nail core 100. The circumferentially interrupted designs of the inner sleeve 28 and the outer sleeve 26 provide a very suitable tool engagement for tensioning or detensioning the spring elements 18, 1018.

Here, the proximal end 112 of the nail core 100 has been fully advanced into the aperture 22, or into the inner sleeve 28. This corresponds to the desired end position of the nail core 100 relative to the sheath element 10.

Finally, it can be clearly seen here that the length L of the nail core 100 corresponds to the axial extension of the wound spring element 18 plus the length of the nail head 20.

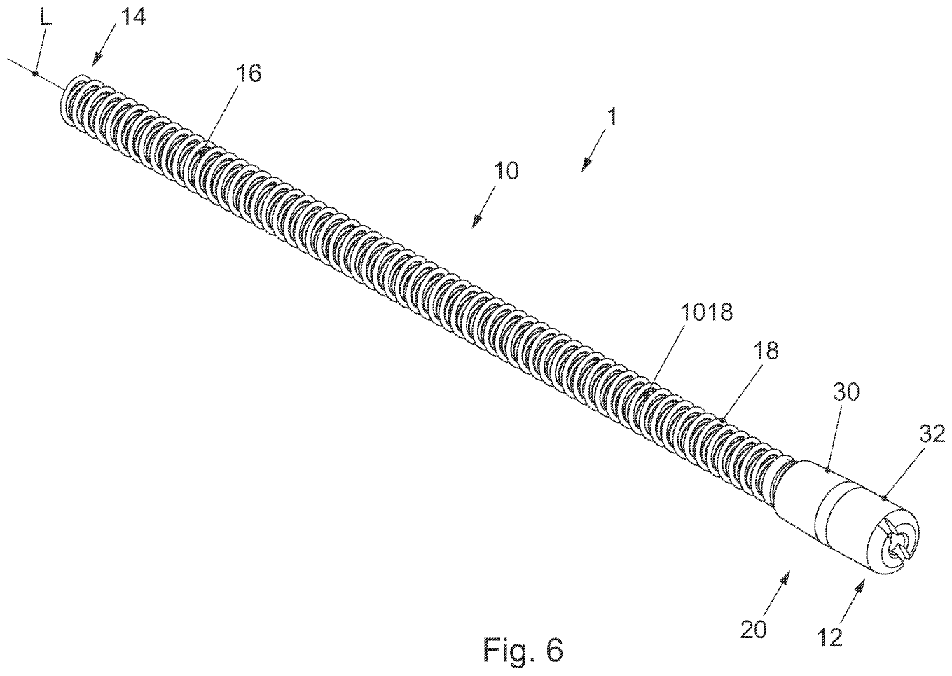

FIG. 6 shows a perspective view of the embodiment of the intramedullary nail 1 according to the invention from FIGS. 1, 2 and 5, which is shown here with the nail core 100 fully inserted, and with an end cap 32 positioned above it.

In other words, initially the situation from FIG. 5 is shown here, whereby here additionally the end cap 32 has been screwed onto the proximal end 12 of the sheath element 10, namely onto the nail head 20. Thus, the intramedullary nail 1 is shown in an implanted or a ready-to-use state.

A suitable tool engagement for operating the end cap 32 is also well visible on the end face of the end cap 32.

Supplementary or in addition to the advantageous embodiments, further developments and examples already discussed, the invention is described below with reference to further specific preferred embodiments, which, however, do not limit the invention to the embodiments described. These embodiments are expressly part of the present description.

Further developments of the advantageous embodiments and examples described above with features of the embodiments described below, just like further developments of the embodiments described below with features of the embodiments and examples described above, expressly form further advantageous embodiments of the invention, and thus form part of the present disclosure.

Embodiment 1: Intramedullary nail for internal stabilization and/or fixing of an elongate hollow body, in particular a bone, wherein the intramedullary nail comprises two separate parts, namely a) a sheath element extending in a longitudinal direction and having a proximal end, a distal end, and a substantially cylindrical interior, wherein an external contour of the sheath element, viewed perpendicularly to the longitudinal direction, comprises an elongate spring element, which is wound in a helical shape starting from the proximal end and with respect to the longitudinal direction, and wherein the extension of the external contour of the sheath element can be reduced by tensioning the elongate spring element in the winding direction and can be increased by detensioning the elongate spring element counter to the winding direction, and b) a substantially rod-shaped nail core which can be inserted into the interior through the proximal end of the sheath element.

Embodiment 2: Intramedullary nail according to the preceding embodiment, wherein the sheath element essentially has the shape of a hollow cylinder.

Embodiment 3: Intramedullary nail according to one or both of the preceding embodiments, wherein the external contour of the sheath element is essentially formed by the wound spring element.

Embodiment 4: Intramedullary nail according to one or more of the preceding embodiments, wherein the spring element is wound in the form of a three-dimensional coil, and in particular in the form of a cylindrical coil.

Embodiment 5: Intramedullary nail according to one or more of the preceding embodiments, wherein the spring element is elastically deformable.

Embodiment 6: Intramedullary nail according to one or more of the preceding embodiments, wherein the spring element forms a three-dimensional coil spring, and in particular a helical spring.

Embodiment 7: Intramedullary nail according to one or more of the preceding embodiments, wherein there is a gap or no gap between adjacent windings of the spring element.

Embodiment 8: Intramedullary nail according to one or more of the preceding embodiments, wherein the spring element is a wire or a strip.

Embodiment 9: Intramedullary nail according to one or more of the preceding embodiments, wherein the sheath element, by expanding its external contour, is braceable against the inner wall of an elongate hollow body to be stabilized.

Embodiment 10: Intramedullary nail according to one or more of the preceding embodiments, wherein the sheath element comprises two elongate spring elements which are arranged one above the other, and which are each wound in a helical shape with respect to the longitudinal direction of the sheath element.

Embodiment 11: Intramedullary nail according to the preceding embodiment, wherein the two spring elements arranged one above the other form a three-dimensional double spiral, in particular a double helix.

Embodiment 12: Intramedullary nail according to one or both of the two preceding embodiments, wherein the two spring elements arranged one above the other are wound in opposite directions or in the same direction.

Embodiment 13: Intramedullary nail according to one or more of the three preceding embodiments, wherein the two spring elements arranged one above the other are wound in opposite directions, and consist of a continuous element which, at the distal end, transitions from a first spring element into a second spring element.

Embodiment 14: Intramedullary nail according to one or more of the preceding embodiments, wherein the nail core is insertable from the outside into a sheath element which itself is already inserted into a hollow body to be stabilized, or which itself is already implanted in a bone.

Embodiment 15: Intramedullary nail according to one or more of the preceding embodiments, wherein the nail core is elastically deformable, in particular elastically bendable.

Embodiment 16: Intramedullary nail according to one or more of the preceding embodiments, wherein the nail core is insertable into a sheath element which comprises a kink or a bend.

Embodiment 17: Intramedullary nail according to one or more of the preceding embodiments, wherein the nail core consists of solid material.

Embodiment 18: Intramedullary nail according to one or more of the preceding embodiments, wherein the nail core comprises a round, in particular a circular, cross-section.

Embodiment 19: Intramedullary nail according to one or more of the preceding embodiments, wherein the nail core comprises a Herzog curvature.

Embodiment 20: Intramedullary nail according to one or more of the preceding embodiments, wherein the proximal end of the sheath element comprises a substantially axial aperture for insertion of the nail core.

Embodiment 21: Intramedullary nail according to one or more of the preceding embodiments, wherein the proximal end of the sheath element comprises a nail head.

Embodiment 22: Intramedullary nail according to the preceding embodiment, wherein the nail head comprises a sleeve or bushing which is rotatable about the longitudinal axis and which is connected to the origin or the end of a spring element.

Embodiment 23: Intramedullary nail according to one or more of the preceding embodiments, wherein the proximal end of the sheath element, in particular a nail head, comprises an end cap on the side facing away from the sheath element.

Embodiment 24: Intramedullary nail according to one or more of the three preceding embodiments, wherein the length of the nail core correlates with the axial extension of the wound spring element plus the length of the nail head.

Embodiment 25: Intramedullary nail according to one or more of the preceding embodiments, wherein the distal end of the nail core comprises a tip which is rounded.

Embodiment 26: Intramedullary nail according to one or more of the preceding embodiments, wherein the proximal end of the nail core is fixable or anchorable in the proximal end of the sheath element, in particular in a nail head.

Embodiment 27: Intramedullary nail according to one or more of the preceding embodiments, wherein the proximal end of the nail core is widened with respect to another radial extension of the nail core.

Embodiment 28: Intramedullary nail according to the preceding embodiment, wherein the widening is realized by the process of forming, impacting or crushing, or by a thread.

Embodiment 29: Intramedullary nail according to one or more of the preceding embodiments, wherein a diameter of the sheath element is reducible to a diameter of the nail core by tensioning a spring element.

Embodiment 30: Intramedullary nail according to one or more of the preceding embodiments, wherein the nail core is inserted into the interior.

Embodiment 31: Intramedullary nail according to one or more of the preceding embodiments, wherein the intramedullary nail further comprises at least one of the features discussed in the above description.

Embodiment 32: Intramedullary nail according to one or more of the preceding embodiments, wherein the intramedullary nail comprises the additional features of at least one of the embodiments set forth in the above description.

Embodiment 33: Intramedullary nail according to one or more of the preceding embodiments, wherein the intramedullary nail is formed according to one of the embodiments set forth in the above description.

Embodiment 34: Intramedullary nail according to one or more of the preceding embodiments, wherein the intramedullary nail comprises the additional features of at least one further preceding embodiment.

Embodiment 35: A nail core for an intramedullary nail according to one or more of the preceding embodiments.

Embodiment 36: Method of internally stabilizing and/or fixing an elongate hollow body, the method comprising the following steps, and in particular comprising the steps in the order listed:
a) Providing an intramedullary nail according to one or more of the preceding embodiments 1 to 34,
b) Optionally, reducing the extension of the external contour of the sheath element by tensioning the elongate spring element in the winding direction,
c) Inserting the sheath element into the hollow body, starting with its distal end,
d) Optionally, increasing the extension of the external contour of the sheath element by detensioning the elongate spring element counter to the winding direction,
e) Optionally, bracing the sheath element against an inner wall of the hollow body by further detensioning the elongate spring element counter to the winding direction,
f) Inserting, starting with its distal end, the nail core into the interior of the sheath element via the proximal end of the sheath element,
g) Optionally, anchoring the nail core in the sheath element, in particular in a nail head.

Embodiment 37: Method of internally stabilizing and/or fixing a bone, the method comprising the following steps, and in particular comprising the steps in the order listed:
a) Providing an intramedullary nail according to one or more of the preceding embodiments 1 to 34,
a1) Optionally, creating an incision point in the bone,
a2) Optionally, drilling out or milling out the medullary canal,
b) Optionally, reducing the extension of the external contour of the sheath element by tensioning the elongate spring element in the winding direction,
c) Inserting, starting with its distal end, the sheath element into the bone, in particular via an incision point in the bone,
d) Optionally, increasing the extension of the external contour of the sheath element by detensioning the elongate spring element counter to the winding direction,
e) Optionally, bracing the sheath element against an inner wall of the bone by further detensioning the elongate spring element counter to the winding direction,
f) Inserting, starting with its distal end, the nail core into the interior of the sheath element via the proximal end of the sheath element,
g) Optionally, anchoring the nail core in the sheath element, in particular in a nail head,
g1) Optionally, closing the proximal end of the sheath element.

Embodiment 38: Method according to one of the two preceding embodiments, wherein the method comprises at least one of the features discussed in the above description, and/or wherein the method is formed according to one of the embodiments set forth in the above description, or comprises at least one feature thereof.

1.-31. (canceled)

32. An intramedullary nail comprising:
a sheath element extending in a longitudinal direction and having a proximal end, a distal end, and a substantially cylindrical interior,
wherein an external contour of the sheath element, viewed perpendicularly to the longitudinal direction, comprises an elongate spring element, which is wound in a helical shape starting from the proximal end and with respect to the longitudinal direction, and
wherein an extension of the external contour of the sheath element is reducible by tensioning the elongate spring element in a winding direction and is increasable by detensioning the elongate spring element counter to the winding direction; and
a substantially rod-shaped nail core insertable into the interior through the proximal end of the sheath element,
wherein the sheath element and the rod-shaped nail core are two separate parts.

33. The intramedullary nail according to claim 32, wherein the sheath element has the shape of a hollow cylinder.

34. The intramedullary nail according to claim 32, wherein the external contour of the sheath element is the wound spring element.

35. The intramedullary nail according to claim 32, wherein the spring element is wound in form of a three-dimensional coil.

36. The intramedullary nail according to claim 32, wherein the spring element forms a three-dimensional coil spring.

37. The intramedullary nail according to claim 32, wherein there is a gap between adjacent windings of the spring element.

38. The intramedullary nail according to claim 32, wherein the spring element is a wire or a strip.

39. The intramedullary nail according to claim 32, wherein the sheath element is, when its external contour is expanded, braceable against an inner wall of an elongate hollow body to be stabilized.

40. The intramedullary nail according to claim 32, wherein the sheath element comprises two elongate spring elements, which are arranged one above the other, and which are each wound in a helical shape with respect to the longitudinal direction of the sheath element.

41. The intramedullary nail according to claim 40, wherein the two spring elements arranged one above the other form a three-dimensional double spiral.

42. The intramedullary nail according to claim 40, wherein the two spring elements arranged one above the other are wound in opposite directions or in the same direction.

43. The intramedullary nail according to claim 40, wherein the two spring elements arranged one above the other are wound in opposite directions, and consist of one continuous element which, at the distal end, transitions from a first spring element into a second spring element.

44. The intramedullary nail according to claim 32, wherein the nail core is insertable from an outside into the sheath element which itself is already inserted into a hollow body to be stabilized, or which itself is already implanted in a bone.

45. The intramedullary nail according to claim 32, wherein the nail core is elastically bendable.

46. The intramedullary nail according to claim 32, wherein the nail core is insertable into the sheath element, which comprises a kink or a bend.

47. The intramedullary nail according to claim 32, wherein the proximal end of the sheath element comprises a substantially axial aperture configured for insertion of the nail core.

48. The intramedullary nail according to claim 32, wherein the proximal end of the sheath element comprises a nail head.

49. The intramedullary nail according to claim 48, wherein the nail head comprises a sleeve, which is rotatable about a longitudinal axis and which is connected to an origin or an end of a spring element.

50. The intramedullary nail according to claim 48, wherein a length of the nail core correlates with an axial extension of the wound spring element plus a length of the nail head.

51. The intramedullary nail according to claim 32, wherein the proximal end of the sheath element comprises an end cap on a side facing away from the sheath element.

52. The intramedullary nail according to claim 32, wherein the distal end of the nail core comprises a rounded tip.

53. The intramedullary nail according to claim 32, wherein the proximal end of the nail core is fixable in the proximal end of the sheath element.

54. The intramedullary nail according to claim 32, wherein the proximal end of the nail core is widened with respect to another radial extension of the nail core.

55. The intramedullary nail according to claim 32, wherein a diameter of the sheath element is reducible to a diameter of the nail core by tensioning the spring element.
